(19) United States
(12) Patent Application Publication
(10) Pub. No.: US 2014/0288269 A1
(43) Pub. Date: Sep. 25, 2014

(54) PROCESS FOR THE PREPARATION OF RANDOM POLYPEPTIDES AND EMPLOYING CIRCULAR DICHROISM AS A GUIDANCE TOOL FOR THE MANUFACTURE OF GLATIRAMER ACETATE

(71) Applicant: Biocon Limited, Bangalore (IN)

(72) Inventors: Srinivas Pullela Venkata, Andhra Pradesh (IN); Anand Khedkar, Bangalore (IN); Nitin Sopanrao Patil, Bangalore (IN); Sandhya Ujire, Bangalore (IN); Amarnath Chatterjee, Bangalore (IN); Ashwini Janakiraman, Bangalore (IN)

(21) Appl. No.: 14/355,504

(22) PCT Filed: Oct. 26, 2012
(86) PCT No.: PCT/IN2012/000708

§ 371 Date: Apr. 30, 2014

(30) Foreign Application Priority Data
Nov. 3, 2011 (IN) 3781/CHE/2011

(51) Int. Cl. C07K 1/04; C07K 1/14

(52) U.S. Cl. CPC C07K 1/045; C07K 1/14
USPC 530/344

(57) ABSTRACT

The present invention discloses novel process for the preparation of mixture of polypeptides comprising L-Glutamaic acid, L-Alanine, L-Tyrosine, and L-Lysine. by employing circular dichroism as a guidance tool.

### FIELD OF THE INVENTION

Novel preparation processes of random polypeptide comprising of the amino acids L-Glutamic acid, L-Alanine, L-Tyrosine and L-Lysine and employing circular dichroism as an analytical tool for the synthesis are disclosed. This mixture of random polymers is used to treat multiple sclerosis as glatiramer in a pharmaceutically acceptable salt form.

### BACKGROUND OF THE INVENTION

Circular Dichroism (CD) spectroscopy is a form of light absorption spectroscopy that measures the difference in absorbance of right- and left-circularly polarized light by a substance. The spectrum obtained due to this phenomenon is called CD spectrum in which the CD signal is represented in terms of millidegrees (mdeg). This phenomenon is exhibited in the absorption bands of optically active chiral molecules. CD spectroscopy has a wide range of applications in many different fields. Most notably, UV CD is used to investigate the secondary structure of proteins. UV/V is CD is used to investigate charge-transfer transitions. This technique does not yield all the information that atomic coordinates, obtained from X-ray crystallography or NMR spectroscopy, would, but is very helpful to look at structural reproducibility in biologics manufacturing. Nearly all molecules synthesized by living organisms are optically active. Nature is dominated by chemical isomers of one handedness over the other (for example, L-amino acids predominate over D-amino acids in most living organisms; D-sugars predominate over the respective L-isomers). Molecules like these, which lack a mirror plane or center of inversion, are chiral and therefore modulate polarized light. Secondary structure of a protein can be determined by CD spectroscopy in the “far-UV” spectral region (200-260 nm). At these wavelengths the chromophore is the peptide bond, and the signal arises when it is located in a regular, folded environment.

Glatiramer is a peptide based polymer composed of four amino acids: L-Glutamaic acid, L-Alanine, L-Tyrosine, and L-Lysine. It's pharmaceutically acceptable salt Glatiramer acetate is approved by FDA and marketed as Copaxone® for the treatment of multiple sclerosis. Copaxone is also known as copolymer-1 and cop-1. Multiple sclerosis is an autoimmune disease affects the brain and central nervous system due to the damage to the myelin sheath of the nerve cells, which results as demyelination of axons. Glatiramer acetate is a synthetic polypeptide analogue of myelin basic protein (MBP). Pharmacologically, Copaxone is a non-interferon and non-steroidal immunomodulator, which arrests the multiple sclerosis aggression. Glatiramer acetate is administrated by subcutaneous injections.

Chemically, glatiramer acetate is designated L-glutamic acid polymer with L-alanine, L-lysine and L-tyrosine, acetate salt. Its structural formula is:

(Glu, Ala, Lys, Tyr)ₓ.xCH₃COOH

(C₅H₉NO₄.C₃H₇NO₂.C₆H₁₄N₂O₂.C₉H₁₁NO₃)ₓ.xC₂H₄O₂

Average molecular weight of glatiramer acetate is 5,000-11,000 daltons and the average molar fractions of the respective amino acids are: 0.141, 0.427, 0.095, and 0.338.

U.S. Pat. No. 3,849,550 describes the observation of MBP arrest in experimental allergic encephalomyelitis (a disease similar to multiple sclerosis) by immunotherapy agents. With continuous endeavours, glatiramer acetate is resulted as an advanced analogue for the treatment of multiple sclerosis with improved safety and efficacy.

U.S. Pat. Nos. 5,800,808; 5,981,589; 6,048,898 describes the process preparation of glatiramer acetate employing the N-carboxyanhydrides (NCAs) derived from alanine, γ-benzyl glutamate, N-trifluoroacetyl lysine, and tyrosine. Following the steps: polymerization, sequential cleavage of the γ-benzyl ester of glutamate and N^{∈}-trifluoroacetyl derivative of lysine, acetate salt formation and final purification. U.S. Pat. No. 6,620,847 describes a process for the preparation of glatiramer acetate using the aqueous piperidine for trifluoroacetyl cleavage of lysine. U.S. Pat. No. 7,049,399 describes the process for preparation of polypeptide-1 using the catalytic transfer hydrogenation for the cleavage of γ-benzyl ester of glutamate. E.P. Pat. No. 1,807,467 describes the processes for preparation of glatiramer using NCAs of alanine, tyrosine, N-t-butoxycarbonyl L-Lysine, and protected glutamic acid, where in the protecting group is selected from γ-methoxybenzyl and γ-benzyl. U.S. Pat. No. 7,495,072 describes the process for the preparation of mixtures of polypeptides using purified hydrobromic acid. The major drawback of all these processes is the generation of impurities, multiple steps of purification and the variability in the secondary structures amongst different batches manufactured using the same process.

Due to the inherent randomness present in GA, routinely used analytical techniques for identification of peptides, like mass-spectroscopy, peptide mapping and chromatography based methods are unable to decipher, unambiguously, the presence of global differences, if any, amongst GA's produced by different synthetic routes, rapidly. Far-UV CD spectra, on the other hand has proved to be a decisive tool that can help in obtaining insights into differences in the peptide ensemble that constitutes the random co-polymer. The Far-UV CD spectrum of GA (FIG. 1; solid line) shows the presence of two distinct secondary structure characteristics, namely random coils (CD absorbance in the wavelength region 195-215 nm) and □-helices (CD absorbance at 222 nm). Hence one may say that the GA is constituted by two ensembles of peptides. Any deviation from this arrangement is picked by the Far-UV CD scan as seen in the figure below,

Thus the Far-UV CD spectrum can be used as guiding tool for designing synthetic routes to obtain the correct ensemble of peptides that constitute the Glatiramer Acetate random Co-polymer.

The instant invention of novel process to synthesise Glatiramer acetate circumvents the batch to batch variability in the secondary structures and proves to be a robust process to manufacture Glatiramer acetate.

### SUMMARY OF THE INVENTION

The present invention describes the novel and robust processes for the preparation of glatiramer acetate. The instant invention demonstrates the process which discloses the deprotection of protected polymer by employing resins and alkali metal alkoxides independently. In one of the embodiments, after the polymerization of NCAs of alanine, tyrosine, N-trifluoroacetyl L-Lysine, and protected glutamic acid, the deprotection of protected L-glutamate moiety and protected l-lysine separately by employing acid resin followed by a suitable base.

In another embodiment, after the polymerization of NCAs of alanine, tyrosine, N-trifluoroacetyl L-Lysine, and protected glutamic acid, the deprotection of protected L-glutamate moiety and protected l-lysine is done in a single step by employing alkali metal alkoxide.

After the deprotection, in both the cases process was proceeded to make acetate salt followed by optional purification.

The disclosed processes are better in ease of handling, operations, and isolations in large scales. The acidic resins employed for the deprotection in the instant invention are less hazardous, easy to handle and better separation from the reaction mixture compared to any other reagents like HBr, H₂SO₄ for cleavage of the protected groups.

Further, the synthesis of Glatiramer acetate was carried out using different kinds of bases which was analysed using various kinds of analytical techniques including size exclusion chromatography and circular dichroism.

Although the molecular weight distribution curves (SEC) of glatiramer acetate synthesised using different kinds of bases exhibited an overlap with the RLD, there was a difference in the Far UV-CD spectra when compared to the RLD.

FIG. 2 shows the overlay of molecular weight distribution (SEC) of generic GA with the RLD.

FIG. 3 shows the comparison of Far-UV CD spectrum with the RLD

Based on RLD data a novel and alternate process was developed to ensure that the molecular weight distribution and Far UV-CD spectra was comparable with RLD.

This invention discloses the use of alkali metal alkoxides, more precisely but not limiting to potassium tert butoxide, sodium methoxide, sodium ethoxide and sodium tert butoxide in the deprotection of Trifluoroacetyl group to synthesise glatiramer acetate.

FIG. 4 shows the overlay of molecular weight distribution (SEC) of generic GA synthesised using alkali metal alkoxides with the RLD.

FIG. 5 shows the comparison of Far-UV CD spectrum with the RLD

### DETAILED DESCRIPTION OF THE INVENTION

The NCA derivatives of protected L-Glutamate, L-alanine, L-Tyrosine, and protected L-Lysine are prepared by following the known procedure. Upon the polmerization, these derivatives produce the corresponding protected copolymer. Deprotection of protecting groups with suitable reagents yields crude glatiramer, further treated with glacial acetic acid and purification leads to get the pure glatiramer acetate.

In one embodiment of the invention, the protected polymer 1 was treated with solid acidic resin with and work up procedures to produce corresponding protected polymer 2 by cleaving the acid labile groups. The reaction proceeded smoothly in short time even in large scales, followed by simple workup and isolation steps. The reaction workup procedure for these resins mediated reactions was very simple when compared to another literature known acids.

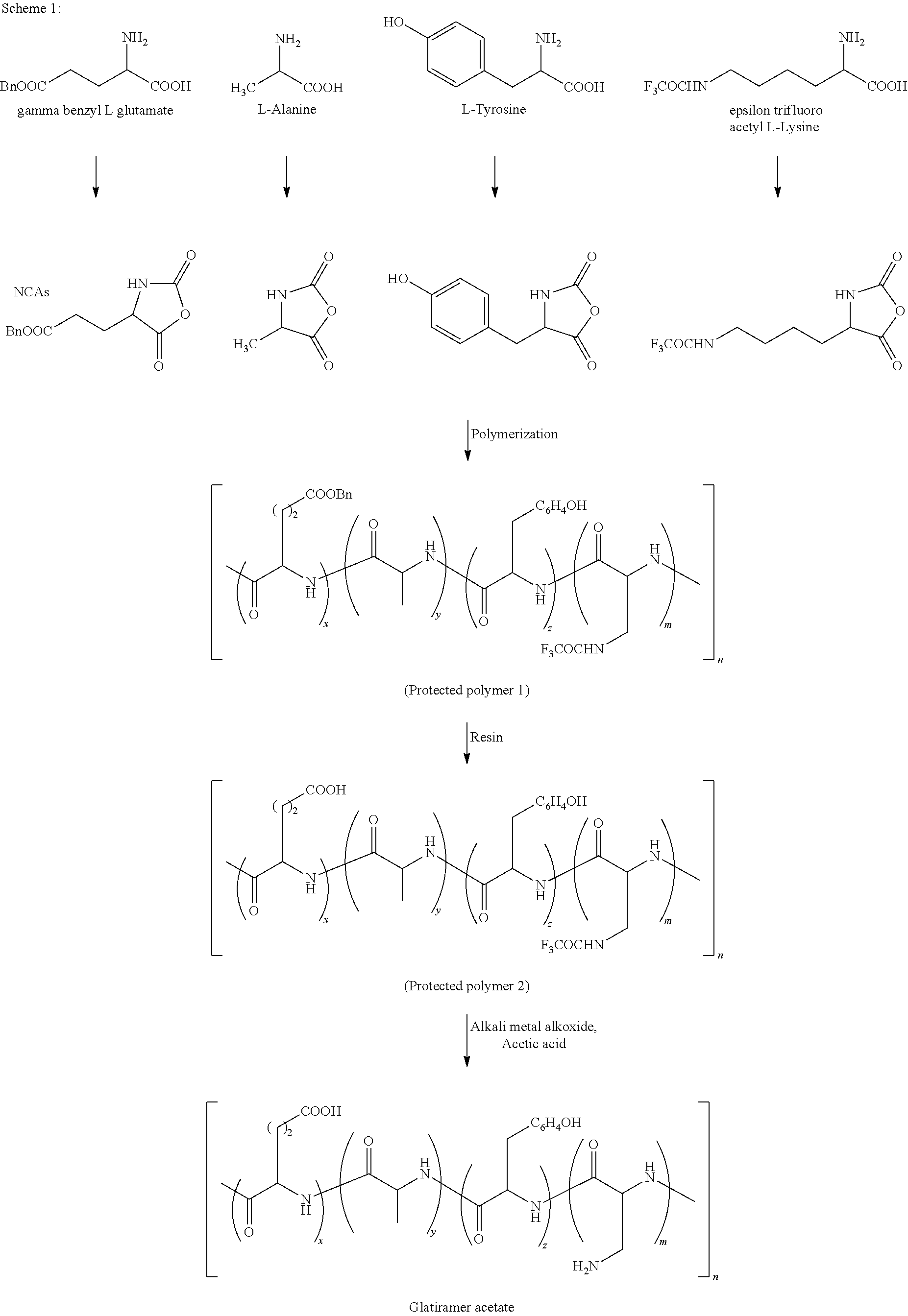

Protected polymer 1 was produced using NCA derivative of γ-benzyl glutamate as one of the components in the polymer, which upon treatment with acidic resin selected from Lewatit K 2629, Diaion UBK 550, Diaion SK 110, Amberlyst-15, or mixture thereof produced the corresponding protected copolymer 2. Interestingly, the same protected polymer 2 was produced by replacing the resin with aluminium chloride, NaI/TMSCI in suitable solvents at appropriate conditions. The solvent was selected from dioxane, THF, acetonitrile, water or mixture thereof. In the next step, base labile protecting groups were cleaved using suitable reagent selected from alkali metal alkoxides, more precisely, potassium tert butoxide, sodium methoxide, sodium ethoxide and potassium tert butoxide, or mixtures thereof. This was followed by adjusting pH to 5.5 with acetic acid and finally purified to obtain glatiramer acetate.

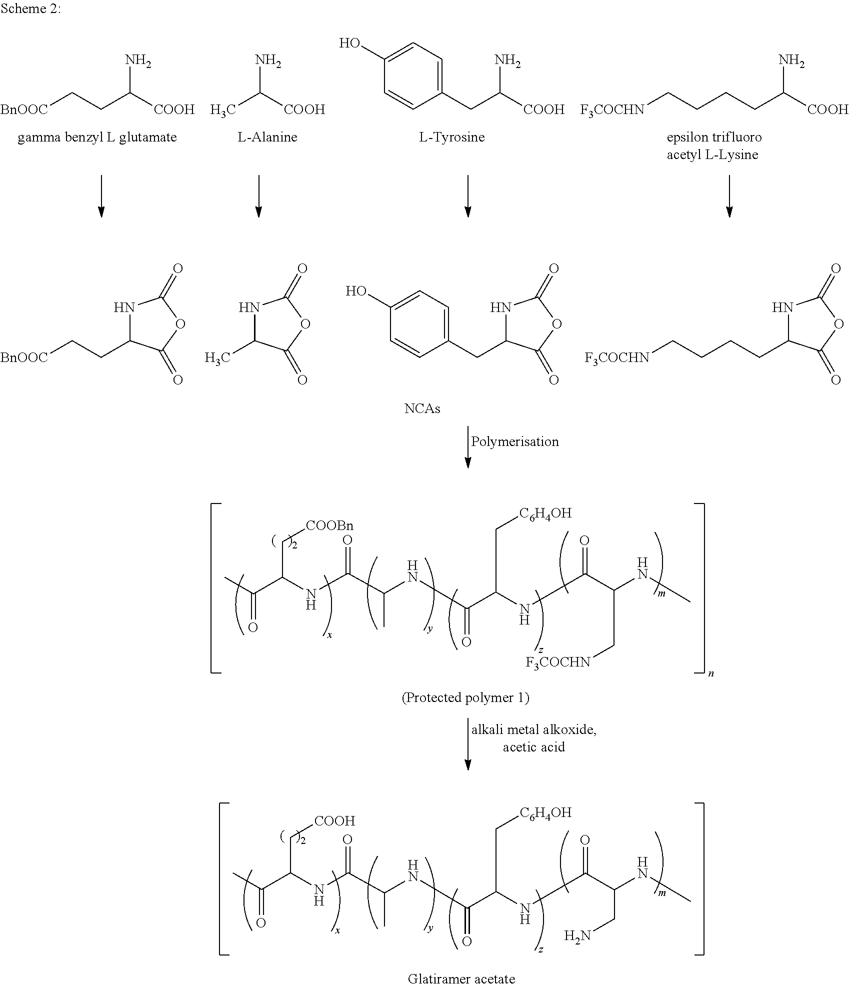

In another embodiment, protected polymer 1 was produced using the NCA derivatives of alkyl glutamate, L-alanine, L-tyrosine, and ε-N-trifluoroacetyl L-Lysine. Alkyl group in alkyl glutamate is selected from C1 to C4 alkanes and optionally protected with a phenyl group. Cleavage of all the protecting groups of the protected polymer 1 was achieved using alkali metal alkoxide in suitable solvents. Further, the pH was adjusted to 5.5 using glacial acetic acid followed by purification to obtain glatiramer acetate.

Glatiramer acetate synthesised using alkali metal alkoxide as a deprotecting reagent exhibited a similar secondary structure profile when compared with the RLD, consistently and with no batch to batch variability.

In yet another embodiment, the deprotection of the base labile group was carried out using a alkali metal alkoxide at a temperature between 20 to 60° C. in a duration of 2 to 72 hrs.

### EXAMPLES

### Example 1

### Preparation of Glatiramer Acetate Using Potassium Tert Butoxide

### Preparation of Protected Polymer 2

Protected copolymer (1 gm) was taken in a mixture of THF and water, to that Lewatit K 2629 resin (1 gm) was added and stirred at 65° C. for 24 h. The resin was filtered through buckner funnel and washed with THF (5 ml).The reaction mass was distilled to 3-4 volume stage and water was added and the precipitated product was filtered and dried in VTD for 24 h at 40-45° C. Yield: 0.6 gm

### Preparation of Glatiramer Acetate

To the stirred solution of protected polymer 2 (0.6 g) in anhydrous methanol (9 ml) was added potassium tertiary butoxide (0.6 g) and stirred for 1 hour. Reaction mass was concentrated under reduced pressure (below 35^{□} C.). To the reaction mass water (0.6 mL) was added and pH was adjusted with Glacial acetic acid to 5.5. Crude glatiramer acetate was isolated by crystallising with acetone. Crystallised solid was filtered and suck dried. Yield: 0.4 g

Crude glatiramer acetate obtained is subjected for gel permeation chromatography for purification.

### Example 2

### Preparation of Glatiramer Acetate Using Sodium Methoxide

### Preparation of Protected Polymer 2

Protected copolymer (1 gm) was taken in a mixture of THF (8 ml) and water (ml), to that Lewatit K 2629 resin (1 gm) was added and stirred at 65° C. for 24 h. The resin was filtered through buckner funnel and washed with THF (5 ml).The reaction mass was distilled to 3-4 volume stage and water was added and the precipitated product was filtered and dried in VTD for 24 h at 40-45° C. Yield: 0.6 gm

### Preparation of Glatiramer Acetate

To the stirred solution of protected polymer 2 (0.6 g) in anhydrous methanol (6 ml) was added a solution of sodium methoxide (0.9 g) in anhydrous methanol (4.5 mL)and stirred for 7 hours. Reaction mass was concentrated under reduced pressure (below 35° C.). To the reaction mass water (0.6 mL) was added and pH was adjusted with Glacial acetic acid to 6. Crude glatiramer acetate was isolated by crystallising with acetone. Crystallised solid was filtered and suck dried. Yield: 0.4 g

Crude glatiramer acetate obtained is subjected for gel permeation chromatography for purification.

### Example 3

### Preparation of Glatiramer Acetate Using TMSCl/NaI Followed by Sodium Methoxide

Protected polymer 1 (20 g) was charged in THF (200 ml) under nitrogen atmosphere, added sodium iodide (1 g) was added followed by trimethylsilyl chloride (20 ml) at room temperature and stirred for 3 h. The reaction mass was quenched after the completion of reaction with water (20 ml). The solids were filtered, washed with water (100 ml) and dried under high vacuum to obtain protected copolymer 2 (10 g).

The resulted protected polymer 2 was suspended in anhydrous methanol (100 ml), solution of sodium methoxide (15 g)in anhydrous methanol (75 ml) was added and stirred at room temperature for 7 h. pH was adjusted after the completion of the reaction to 6 with glacial acetic acid, and the mass was purified to obtain glatiramer acetate (6 g).

### Example 4

### Preparation of Glatiramer Acetate from Protected Polymer 3

Solution of sodium methoxide (1.5 g) in anhydrous methanol (7.5 ml) was added to protected copolymer 3 (1 g) in anhydrous methanol (10 ml) at room temperature and stirred for 7 h. pH was adjusted to 5 after completion of the reaction with glacial acetic acid. The resulted mass was purified to obtain glatiramer acetate (0.6 g).

The Far UV CD spectra, of Glatiramer acetate synthesised using the processes described in examples 1-4 exhibits the presence of random coils in the wavelength region 195-215 nm and alpha helices in the wavelength region of 222 nm. (FIG. 5)

1. A process for preparing a polypeptide containing L-Glutamate, L-Alanine, L-Tyrosine, and L-Lysine employing circular dichroism as a guidance tool, the method comprising;
i. polymerizing an N-carboxyanhydride (NCA) derivative of a protected L-Glutamate, L-Alanine, optionally protected L-Tyrosine, and protected L-Lysine,
ii. optionally treating with an acidic resin in a solvent,
iii. treating with base in solvent,
iv. adjusting the pH with acetic acid, and
v. isolating glatiramer acetate.

2. The process of claim 1, wherein the protected L-glutamate NCA derivative is γ-alkyl ester of L-glutamic acid.

3. The process of claim 2, wherein the γ-alkyl ester is an ester of an optionally protected γ-methyl, γ-ethyl, γ-propyl, γ-butyl, or mixture thereof.

4. The process of claim 3, wherein the optionally protected γ-methyl is γ-benzyl.

5. The process of claim 1, wherein the optionally protected L-tyrosine is L-tyrosine, acetyl-L-tyrosine, or a mixture thereof.

6. The process of claim 1, wherein the protected L-lysine is epsilon N trifluoroacetyl L Lysine.

7. The process of claim 1, wherein the acidic resin is Lewatit K 2629, Diaion UBK 550, Diaion SK 110, or Amberlyst-15.

8. The process of claim 7, wherein the reaction temperature is 30 to 70° C.

9. The process of claim 1, wherein the base is an alkali metal alkoxide of the formula MOR, where M is an alkali metal, and R is CₙHₙ₊₁, wherein n is 1 to 10.

10. The process of claim 9, wherein the suitable base is sodium methoxide, sodium tert-butoxide, potassium tert butoxide sodium ethoxide, or a mixture thereof.

11. The process of claim 1, wherein the suitable solvent is anhydrous methanol, ethanol, tert butanol, n-butanol, isopropyl alcohol, n-propyl alcohol, or a mixture thereof.

12. The process of claim 11, wherein the moisture content of the solvent in step (iii) does not exceed 0.5%.

13. The process of claim 12, wherein the moisture content of the solvent in step (iii) is less than 0.1%.

14. The process of claim 1, wherein the reaction temperature is between −20 to 60° C.

15. The process of claim 1, wherein the reaction time is 2 to 72 h.

16. Process for preparing a polypeptide containing L-Glutamate, L-Alanine, L-Tyrosine, and L-Lysine employing circular dichroism as a guidance tool, the method comprising;
i. polymerizing an N-carboxyanhydride (NCA) derivative of a protected L-Glutamate, L-Alanine, an optionally protected L-Tyrosine, and a protected L-Lysine,
ii. treating with alkali metal alkoxide in a solvent,
iii. adjusting the pH with acetic acid, and
iv. isolating glatiramer acetate.

17. The process of claim 16, wherein the protected L-glutamate NCA derivative is a γ-alkyl ester.

18. The process of claim 16, wherein the γ-alkyl ester is an ester of an optionally protected γ-methyl, γ-ethyl, γ-propyl, γ-butyl, or mixture thereof.

19. The process of claim 18, wherein the optionally protected γ-methyl is γ-benzyl.

20. The process of claim 16, wherein the optionally protected L-tyrosine is L-tyrosine, acetyl-L-tyrosine, or a mixture thereof.

21. The process of claim 16, wherein the protected L-lysine is epsilon N trifluoroacetyl L Lysine.

22. The process of claim 16, wherein the alkali metal alkoxide is of the formula MOR, where M is alkali metal, and R is CₙHₙ₊₁, wherein n is 1 to 10.

23. The process of claim 16, wherein the suitable solvent in step (ii) is anhydrous methanol, ethanol, tert butanol, n-butanol, isopropyl alcohol, n-propyl alcohol, or a mixture thereof.

24. The process of claim 16, wherein the moisture content of the solvent in step (ii) does not exceed 0.5%.

25. The process of claim 24, wherein the moisture content of the solvent in step (ii) is less than 0.1%.

26. The process of claim 16, wherein the reaction temperature is −20 to 60° C.

27. The process of claim 16, wherein the reaction time is 2 to 72 h.
